(19) United States
(12) Patent Application Publication
(10) Pub. No.: US 2013/0259921 A1
(43) Pub. Date: Oct. 3, 2013

(54) Treatment Of Diabetic Patients With A Stent And An Adjunctive Drug Formulation

(71) Applicant: Syed F.A. Hossainy, Hayward, CA (US)

(72) Inventor: Syed F.A. Hossainy, Hayward, CA (US)

(73) Assignee: Abbott Cardiovascular Systems Inc., Santa Clara, CA (US)

(21) Appl. No.: 13/436,303

(22) Filed: Mar. 30, 2012

(51) Int. Cl. A61K 31/573; A61K 31/439; A61K 9/127; A61M 25/10; A61M 25/00; A61K 31/337; A61P 9/00; B82Y 5/00

(52) U.S. Cl. 424/450; 514/449; 514/180; 514/291; 604/509; 604/508; 977/773; 977/906

(57) ABSTRACT

Embodiments of the present invention include methods of treating, preventing, or ameliorating a vascular disease and/or disorder in a diabetic or pre-diabetic patient. The methods include implanting a stent in a vascular region in a diabetic patient, and during the implantation procedure, delivering a drug formulation from a source other than the stent to the vascular region. The stent may be a bare metal stent, or a drug eluting stent, such as a metal stent having a coating including a drug. The drug may be everolimus, sirolimus, or a combination thereof. The drug formulation may include dexamethasone, paclitaxel, or a combination thereof.

### BACKGROUND OF THE INVENTION

1. Field of the Invention

This invention relates to methods of treating vascular disease in diabetic patients.

2. Description of the State of the Art

Until the mid-1980s, the accepted treatment for coronary atherosclerosis, i.e., narrowing of the coronary artery(ies), was coronary by-pass surgery. While being quite effective and having evolved to a relatively high degree of safety for such an invasive procedure, by-pass surgery still involves potentially serious complications and in the best of cases an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The re-opening of the artery by an inflatable balloon is also referred to as “dilatation” of the artery. The procedure was relatively non-invasive, took a very short time compared to by-pass surgery, and the recovery time was minimal. However, PTCA brought with it other problems such as vasospasm and elastic recoil of the stretched arterial wall which could undo much of what was accomplished and, in addition, created a new problem, restenosis, the re-clogging of the treated artery due to neointimal hyperplasia.

The next improvement, advanced in the mid-1980s, was the use of a stent to maintain the diameter of the artery after PTCA. This for all intents and purposes put an end to vasospasm and elastic recoil, but did not entirely resolve the issue of restenosis. That is, prior to the introduction of stents, restenosis occurred in from about 30 to 50% of patients undergoing PTCA. Stenting reduced this to about 15 to 20%, a substantial improvement, but still more restenosis than desirable. For diabetic patients, the incidences of restenosis as well as major cardiac events were significantly higher than non-diabetics patients with stenting.

In 2003, drug-eluting stents or DESs were introduced. The drugs employed with the DES are cytostatic compounds, that is, compounds that curtail the proliferation of cells that results in restenosis. The occurrence of restenosis has been reduced to about 5 to 7%, a very improved figure. However, based upon the studies to date, the rate of restenosis with DES is remains higher for diabetic patients than non-diabetic patients. Thus, there is a need for improved methods for treating vascular diseases and disorders, particularly in diabetic and pre-diabetic patients.

### SUMMARY OF THE INVENTION

The present invention is directed to methods of treating, preventing, or ameliorating vascular diseases and disorders in patients who are diabetic or pre-diabetic who are in need of treating, preventing, or ameliorating a vascular disease and/or disorder. The methods involve the implantation of a stent in the patient, and delivery (administration) of a drug formulation from a source other than the stent to the patient. The drug formulations include, but are not limited to, dexamethasone, paclitaxel, or a combination thereof. In some embodiments, the patient is identified as having diabetes or a pre-diabetic condition.

In an aspect of the invention, implanting the stent includes, but is not limited to, delivery of the stent to the vascular region and deployment of the stent at the vascular region.

In an aspect of the invention, the drug formulation administration includes, but is not limited to, administration by a balloon catheter, a catheter, or a guide catheter.

In an aspect of the invention, the stent does not comprise a drug.

In an aspect of the invention, the stent is a bare metal stent.

In an aspect of the invention, the drug formulation includes, but is not limited to, nano-particles, micelles, nano-vesicles, polymersomes, or any combinations thereof which carry and deliver the dexamethasone, paclitaxel, or a combination thereof.

In an aspect of the invention, the drug formulation includes, but is not limited to, nano-particles, the nano-particles comprising poly(lactide), poly(lactide-co-glycolide), or a combination thereof.

In an aspect of the invention, the drug formulation comprises nano-vesicles, the nano-vesicles being liposomes, liposomes with ceramide, or both.

In an aspect of the invention, the drug formulation comprises is a viscous fluid which carries and delivers the dexamethsaone, paclitaxel, or a combination thereof.

In an aspect of the invention, the drug formulation includes, but is not limited to, poly(vinyl alcohol), hydroxypropyl methylcellulose, carboxymethyl cellulose, hyaluronic acid, polyvinylpyrrolidone, polyethylene glycol, polyethylene oxide, and combinations thereof.

In an aspect of the invention, the stent is a DES.

In a further aspect of the invention, the drug of the DES is selected from the group consisting of rapamycin (sirolimus), Biolimus A9, deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, 40-epi-(N1-tetrazolyl)-rapamycin, and combinations thereof.

In an aspect of the invention, the drug formulation is administered by a bolus administration, by an infusion, or by intermittent administration.

In an aspect of the invention, the drug formulation is administered and/or the administration begins within 30 to 90 minutes prior to the insertion of the stent delivery device into the patient.

In an aspect of the invention, the drug formulation is administered and/or the administration begins within 5 to 75 minutes prior to the insertion of the stent delivery device into the patient.

In an aspect of the invention, the drug formulation is administered and/or the administration begins within 15 minutes prior to the insertion of the stent delivery device into the patient.

In an aspect of the invention, the drug formulation is administered or the administration begins during the stent deployment.

In an aspect of the invention, the drug formulation is administered after the stent deployment.

In an aspect of the invention, the time period of drug administration at least partially overlaps the time period of stent deployment.

In an aspect of the invention, at least 30% of the time period of drug administration overlaps the time period of stent deployment.

In an aspect of the invention, the vascular disease in the patient is a stenosis or a restenosis.

### BRIEF DESCRIPTION OF THE DRAWINGS

FIG. 1 is an elevational view, partially in section, of an exemplary stent which is mounted on a delivery catheter and disposed within a damaged artery.

FIG. 2 is an elevational view, partially in section, similar to that shown in FIG. 1 wherein the stent is expanded within a damaged artery.

FIG. 3 is an elevational view, partially in section, depicting the expanded stent within the artery after withdrawal of the delivery catheter.

### DETAILED DESCRIPTION OF THE INVENTION

Use of the term “herein” encompasses the specification, the abstract, and the claims of the present application.

Use of the singular herein includes the plural and vice versa unless expressly stated to be otherwise, or obvious from the context that such is not intended. That is, “a” and “the” refer to one or more of whatever the word modifies. For example, “a drug” includes one drug, two drugs, etc. Likewise, “the polymer” may refer to one, two or more polymers, and “the device” may mean one device or a plurality of devices. By the same token, words such as, without limitation, “polymers” and “devices” would refer to one polymer or device as well as to a plurality of polymers or devices unless, again, it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. In general, but with the preceding discussion in mind, a numerical value herein that is modified by a word of approximation may vary from the stated value by ±15%, unless expressly stated otherwise.

As used herein, any ranges presented are inclusive of the end-points. For example, “a duration of time between 10 and 75 minutes” or “a duration of time from 10 to 75 minutes” includes 10 minutes and 75 minutes, as well as any specific duration of time in between 10 minutes and 75 minutes.

As used herein, a “cardiovascular disease” is a disease, condition, or disorder that impacts the heart, circulatory system, or both the heart and the circulatory system. The circulatory system is the lymphatic system and the cardiovascular system. The lymphatic system distributes lymph. The cardiovascular system is a system of blood vessels, primarily arteries and veins, which transport blood to and from the heart, brain and peripheral organs such as, without limitation, the arms, legs, kidneys and liver. The coronary system supplies blood to and from the heart, and includes the coronary artery system, which supplies blood to the heart. The carotid system supplies blood to and from the brain, and includes the carotid artery system, which supplies blood to the brain. The peripheral vascular system carries blood to (primarily via arteries) and from (primarily via veins) the peripheral organs such as, without limitation, the hands, legs, kidneys and liver. The coronary system, carotid system, and the peripheral vascular system are part of the cardiovascular system.

As used herein, a “vascular disease” refers to a disease, condition, or disorder that impacts the circulatory system. In particular “vascular disease” includes a disease, disorder, or condition of the coronary system, the carotid system, and/or the peripheral vascular system.

“Vascular diseases” are a subset of “cardiovascular diseases.”

Examples of cardiovascular diseases include diseases of the heart which include, but are not limited to, heart valve disease, arrhythmia, heart failure, and congenital heart disease, and vascular diseases, which include, but are not limited to atherosclerosis, thrombosis, restenosis, hemorrhage, vascular dissection or perforation, vulnerable plaque, chronic total occlusion, claudication, anastomotic proliferation for vein and artificial grafts, peripheral artery disease, carotid artery disease, coronary artery disease, aneurysm, renal (kidney) artery disease, Raynaud's disease, buerger's disease (a.k.a. thromboangiitis obliterans), peripheral venous disease, varicose veins, blood clots in the veins, blood clotting disorders, and lymphdema.

As used herein, a “drug” refers to a substance that, when administered in a therapeutically effective amount to a patient suffering from a disease, disorder, or condition, has a therapeutic beneficial effect on the health and well-being of the patient. A therapeutic beneficial effect on the health and well-being of a patient includes, but is not limited to: (1) curing the disease, disorder, or condition; (2) slowing the progress of the disease, disorder, or condition; (3) causing the disease, disorder, or condition to retrogress or to be in remission; or, (4) alleviating, ameliorating or both alleviating and ameliorating one or more symptoms of the disease, disorder, or condition.

As used herein, a “drug” also includes any substance that when administered to a patient, known or suspected of being particularly susceptible to a disease, disorder, or condition, in a prophylactically effective amount, has a prophylactic beneficial effect on the health and well-being of the patient. A prophylactic beneficial effect on the health and well-being of a patient includes, but is not limited to: (1) preventing or delaying on-set of the disease, disorder, or condition in the first place; (2) maintaining a disease, disorder, or condition at a retrogressed level once such level has been achieved by a therapeutically effective amount of a substance, which may be the same as or different from the substance used in a prophylactically effective amount; or, (3) preventing or delaying recurrence of the disease, disorder, or condition after a course of treatment with a therapeutically effective amount of a substance, which may be the same as or different from the substance used in a prophylactically effective amount, has concluded.

As used herein, “drug” also refers to pharmaceutically acceptable, pharmacologically active derivatives of those drugs specifically mentioned herein, including, but not limited to, salts, esters, amides, hydrates, solvates, and the like.

As used herein, the phrase “drug is X” refers also to pharmaceutically acceptable, pharmacologically active derivatives of the drug X, such as, but not limited to, salts, esters, amides, hydrates, solvates, and the like. As a non-limiting an example, “the drug is dexamethasone” would also encompass dexamethasone acetate.

As used herein, a “drug formulation” is a drug in combination with other materials, referred to as excipients. Excipients are non-toxic, and are typically, but not always, inert, that is the excipients themselves are not drugs. Excipients typically perform a function such as acting as a binder for the drug, a carrier or a diluent for the drug, a permeation enhancer, or an antioxidant or stabilizer for the drug. In some cases vitamins and minerals, which may have therapeutic effects themselves, may also be used as an excipients. One of skill in the art can readily determine if a vitamin, mineral, or other substance is being used as an excipient in a drug formulation, or if the vitamin, mineral, or other substance is a drug in the drug formulation. Unlike a solvent, which may be removed from the drug formulation, an excipient is not removed, but remains part of the formulation. A drug formulation may be a final dosage form for administration to the patient, such as a tablet, capsule, or syrup for oral administration, or the drug formulation require further combination with other materials or excipients. Non-limiting examples of formulations which require further combination are powders that are reconstituted or blended with water for oral administration, or with sterile water or solution for injection.

As used herein, a “solvent” refers to a substance capable of dissolving, partially dissolving, dispersing, suspending, or any combination thereof, a substance to form a uniform dispersion, solution, or suspension, with or without agitation, at a selected temperature and pressure. The substance may be a solid, semi-solid, a liquid, a gas, or a supercritical fluid. A solvent herein may be a blend of two or more such substances.

A “normal saline solution,” is a saline solution that is essentially isotonic with blood. Saline solutions are those that contain a salt or salts, typically sodium chloride.

As used herein, a “polymer” refers to a molecule comprised of repeating “constitutional units.” The constitutional units derive from the reaction of monomers. The constitutional units themselves can be the product of the reactions of other compounds. As a non-limiting example, ethylene (CH₂═CH₂) is a monomer that can be polymerized to form polyethylene, CH₃CH₂(CH₂CH₂)ₙCH₂CH₃ (where n is an integer), wherein the constitutional unit is —CH₂CH₂—, ethylene having lost the double bond as the result of the polymerization reaction. A polymer may be derived from the polymerization of two or more different monomers and therefore may comprise two or more different constitutional units. Such polymers are referred to as “copolymers.” “Terpolymers” are a subset of “copolymers” in which there are three different constitutional units. Those skilled in the art, given a particular polymer, will readily recognize the constitutional units of that polymer and will readily recognize the structure of the monomer from which the constitutional units derive. Polymers may be straight chain, branched chain, star-like or dendritic. One polymer may be attached (grafted) onto another polymer. The constitutional units of polymers may be randomly disposed along the polymer chain, may be present as discrete blocks, may be so disposed as to form gradients of concentration along the polymer chain, or a combination thereof. Polymers may be cross-linked to form a network.

As used herein, a polymer has a chain length of 50 constitutional units or more, and those compounds with a chain length of fewer than 50 constitutional units are referred to as “oligomers.”

As used herein, the terms “biodegradable,” “bioerodable,” “bioabsorbable,” “degraded,” “eroded,” “absorbed,” and “dissolved,” are used interchangeably, and refer to a substance that is capable of being completely or substantially completely, degraded, dissolved, eroded, or any combination thereof over time when exposed to physiological conditions (pH, temperature, enzymes and the like), and can be gradually eliminated by the body, or that can be degraded into fragments that can pass through the kidneys. Conversely, “biostable” refers to a substance that is not biodegradable.

As used herein, a material that is described as a layer, a film, or a coating “disposed over” a substrate refers to deposition of the material directly or indirectly over at least a portion of the surface of that substrate. “Directly deposited” means that the material is applied directly to the surface of the substrate. “Indirectly deposited” means that the material is applied to an intervening layer that has been deposited directly or indirectly over the substrate. The terms “film,” and “coating” are used interchangeably herein. A coating may have multiple layers, and each layer may be applied by multiple applications of coating material. Layers typically differ from each other in the type of materials, the ratio of materials, or both the type of and the ratio of materials applied to form the layer. Materials may migrate from one layer to another layer during the coating application process, after the coating has been formed, or both during the coating application process and after the coating has been formed.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, self-expandable stents, balloon-expandable stents, stent-grafts, and grafts.

With respect to an implantable medical device, the “outer surface” is meant any surface however spatially oriented that is in contact with bodily tissue or fluids.

With respect to an implantable medical device, a “device body” refers to an implantable medical device in a fully formed utilitarian state with an outer surface to which no coating or layer of material different from that of which the device itself is manufactured has been applied.

One type of implantable medical device is a stent. Stents are implantable medical devices that are generally cylindrically shaped, and function to hold open, and sometimes expand, a segment of a blood vessel or other lumen or vessel in a patient's body when the vessel is narrowed or closed due to diseases or disorders including, without limitation, coronary artery disease, carotid artery disease and peripheral vascular disease. A stent can be used in, but is not limited to use in, neuro, carotid, coronary, pulmonary, renal, biliary, iliac, femoral and popliteal, and other peripheral vasculatures, as well as other bodily lumens. A stent can be used in the treatment or prevention of cardiovascular diseases and disorders, including vascular diseases and disorders, as well as other diseases and disorders. For a stent, the “outer surface” includes the luminal surface which faces the lumen interior, the abluminal surface which faces the lumen wall, and sidewall surfaces, if present, which connect the abluminal and luminal surfaces.

A bare metal stent (BMS), which, as the name implies, is a fully-formed usable stent that has not been coated with a layer of any material different from the metal of which it is made on any surface that is in contact with bodily tissue or fluids. Similarly, stents may be formed from other materials, or a combination of other materials and a polymer, and not have any coatings disposed over the outer surface.

Another category of medical devices are insertable medical devices. “Insertable medical devices” include any type of appliance that is totally or partly introduced, surgically or medically, into a patient's body or by medical intervention into a natural orifice, but the device does not remain in the patient's body after the procedure.

A “catheter” is a thin, flexible tube for insertion into a natural body cavity, duct, or vessel, to introduce or remove fluid, to distend the vessel, and/or to hold open the vessel or cavity. Catheters may be insertable devices, or may be implanted for several hours or days.

A “vascular catheter” is an insertable medical device. A vascular catheter is a thin, flexible tube with a manipulating means at one end, which remains outside the patient's body, and an operative device at or near the other end, which is inserted into the patient's artery or vein. The catheter may be used for the introduction of fluids, often containing drugs, to the target site. The catheter may be used to deliver a stent to the target site, or may be used to deliver a balloon used in angioplasty. The catheter may perform multiple functions.

As used herein, a “guide catheter” refers to a catheter through which a balloon catheter used in angioplasty may be inserted. A guide catheter may be advanced close to a region to be treated and then a guidewire a balloon catheter may be advanced through the guide. Fluids or other materials such as radio-opaque agents used for visualization may be delivered through a guide catheter.

As used herein, a “balloon” refers to a relatively thin, flexible material, forming a tubular membrane, and is usually associated with a vascular catheter. When positioned at a particular location in a patient's vessel can be expanded or inflated to an outside diameter that is essentially the same as the inside or luminal diameter of the vessel in which it is placed. Balloons may be inflated using a liquid medium such as water or normal saline solution. Non-limiting examples of suitable balloon materials polyester, PEBAX® (polyether block amide block copolymers, Arkema), polyurethanes, poly(tetra-fluoroethylene) (aka PTFE, and TEFLON®, DuPont Co., Wilmington, Del.), nylon, and DACRON® (DuPont Co.).

A “balloon catheter” refers to a medical device which is a system of a catheter with a balloon at the end of the catheter.

A “balloon” of a “balloon catheter” may be used to perform one or more of the following functions: dilate a vessel (“a dilatation balloon”); deliver drug or other substances to a vessel; and expand a stent that has been mounted over the balloon.

An “introducer sheath” is a tube inserted into the body and allows access of other instruments into parts of the body, such as, without limitation, the trachea, a vein, or an artery.

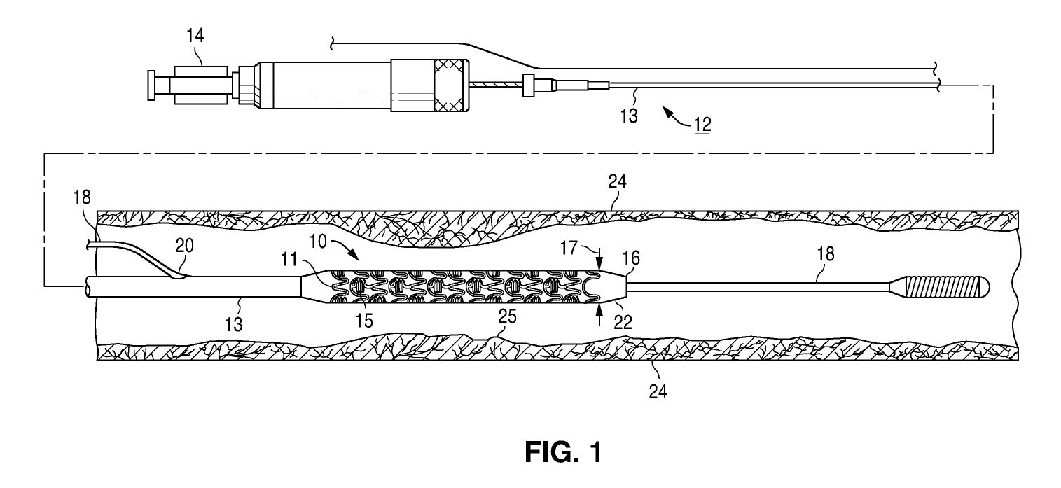

A typical implantation of a stent is described in the following paragraphs. FIG. 1 generally depicts a stent 10, mounted on a catheter assembly 12 which is used to deliver the stent 10 and implant it in a body lumen, such as a blood vessel 24. The non-limiting example of a stent 10 that is shown in FIG. 1 comprises a plurality of radially expandable cylindrical rings 11 disposed generally coaxially and interconnected by undulating links 15 disposed between adjacent cylindrical rings 11. The combination of cylindrical rings 11 and links 15 form the stent 10 body, that is the device body of the stent (also referred to as the scaffolding), which supports the vessel once deployed. The catheter assembly 12 includes a catheter shaft 13 which has two ends, a first end 14 and a second end 16. The catheter assembly 12 is configured to advance through the patient's vascular system by advancing over a guide wire by any of the well known methods, including a rapid exchange catheter system, such as the one shown in FIG. 1. Another well known method for stent delivery is an over the wire system.

Catheter assembly 12 as depicted in FIG. 1 is of the well-known rapid exchange type which includes an RX port 20 where the guide wire 18 will exit the catheter from a lumen, which is a passageway or cavity, in the shaft 13. The distal end of the guide wire 18 exits the catheter second end 16 so that the catheter advances along the guide wire on a section of the catheter between the RX port 20 and the catheter second end 16. If the stent 10 is of the balloon-expandable type, the stent is mounted on a balloon 22 and is crimped tightly thereon so that the stent 10 and balloon 22 present a low profile diameter for delivery through the arteries. Alternatively, a self-expanding stent configuration as is well known in the art may be used.

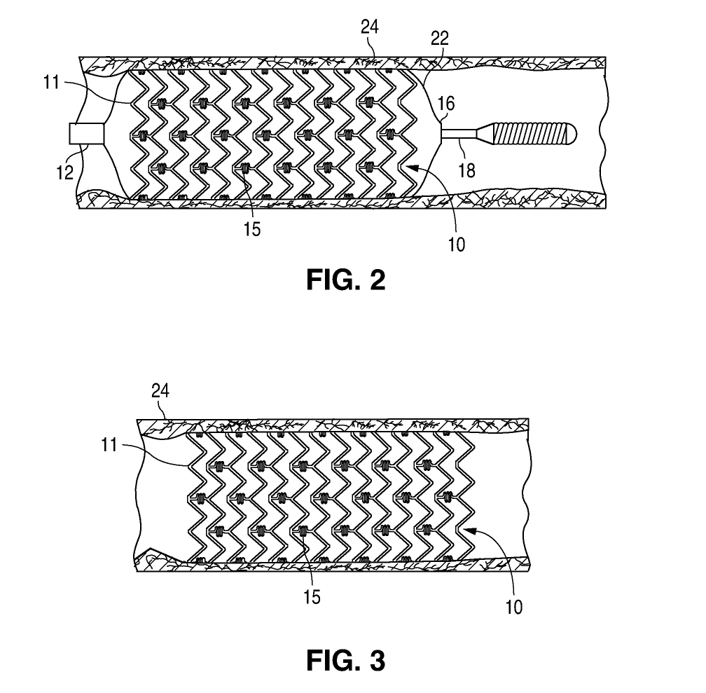

As shown in FIG. 1, a partial cross-section of an artery 24 is shown with a small amount of plaque 25 that has been previously treated by a repair procedure. A stent 10 may be used to repair a diseased or damaged arterial wall which may include the plaque 25 as shown in FIG. 1, or a dissection, or a flap which are commonly found in the coronary arteries, carotid arteries, peripheral arteries and other vessels. In a typical procedure to implant stent 10, the guide wire 18 is advanced through the patient's vascular system by well known methods so that the distal end of the guide wire is advanced past the plaque or diseased area 25. The introduction of the stent into the body and transport to a region that is to be treated is referred to herein as “delivery.” Once the stent 10 has been delivered to the region to be treated, the stent delivery catheter assembly 12 is advanced over the guide wire 18 so that the stent 10 is positioned in the target area. The balloon 22 is inflated by well known means so that it expands radially outwardly and in turn expands the stent 10 radially outwardly until the stent 10 is apposed to the vessel wall. The radial expansion of the stent, by a balloon or otherwise, until the stent is apposed to the vessel wall is referred to herein as “deployment” of the stent. The balloon 22 is then deflated and the catheter withdrawn from the patient's vascular system. The guide wire 18 typically is left in the lumen for post-stent implantation procedures, if any, and subsequently is withdrawn from the patient's vascular system. A lumen in the catheter shaft 13 may be used to deliver fluids, potentially including a drug, to the site, such as the site of plaque 25. As depicted in FIGS. 2 and 3, the balloon 22 is fully inflated with the stent 10 expanded and pressed against the vessel wall, and in FIG. 3, the implanted stent 10 remains in the vessel after the balloon 22 has been deflated and the catheter assembly 12 and guide wire 18 have been withdrawn from the patient. As used herein, “implantation” of a stent refers to the delivery and deployment of the stent.

As obvious form the preceding discussion, a balloon, a catheter, and a stent perform different functions. A stent may be crimped to a smaller diameter for delivery, and then the stent may be subsequently deployed by being allowed to expand if self-expanding, or is expanded by a balloon or other device, to a large diameter. The expanded stent is capable of supporting a bodily lumen for an extended period of time. In contrast, a balloon has a wall thickness that is so thin that the tubular membrane cannot support a load at a given diameter unless inflated with a fluid. Furthermore, a balloon is a transitory device that is inserted in the patient's body for only a limited time for the purpose of performing a specific procedure or function. As a non-limiting example, dilatation balloons used to expand a vessel wall, and optionally open an occluded vessel, are not implanted, but are removed from the body at the end of the procedure. Catheters have a shaft which is similar to a stent in that most stents and catheter shafts are tubular or cylindrical in shape. However, a catheter shaft is not designed to be radially expandable. In addition, a vascular catheter has a much larger (a factor of 10 or greater) length to diameter ratio than a stent.

As discussed previously, the use of stents has reduced the incidence of restenosis, but to a lower extent in diabetic patients. For example, one study found that after a percutaneous cardiac intervention, such as PTCA, followed by the implantation of a bare metal stent, the rate of restenosis was 30% for diabetic patients compared to 20% for non-diabetic patients. Another study involving implantation of a drug-eluting stent (DES), found a rate of 14.6% restenosis in non-diabetics, but 20.9% for diabetic patients. In addition, diabetic patients are more likely to experience major adverse cardiac events (MACE) after PTCA with stenting. In general, diabetics are more than twice as likely as non-diabetics to have a heart attack or stroke, and 2 out of 3 diabetics die from cardiovascular disease (American Diabetes Association). Hyperglycemia, independent of whether or not a person has been diagnosed with diabetes, is a risk-factor for cardiovascular events. It is interesting to note that the risk factors for cardiovascular disease and diabetes significantly overlap. Some have gone as far as postulating that both are part of the “metabolic syndrome.”

Diabetic patients are those individuals suffering from diabetes mellitus, often referred to as just “diabetes,” a group of metabolic diseases. Diabetes may be type 1, previously referred to as juvenile diabetes, in which an individual is unable to produce insulin. Type 1 diabetes may also be called insulin dependent diabetes. Type 2 diabetes results from an insulin level which is too low, or an inability to utilize insulin, referred to as “insulin resistance.” As used herein, a person may be diagnosed as diabetic if at least one of the following applies:
(1) fasting plasma glucose level is greater than or equal to 7.6 mmol/L (126 mg/dL);
(2) plasma glucose level is greater than or equal to 11.0 mmol/L (200 mg/dL) 2 hours after a 75 gram oral glucose load (standard glucose tolerance test);
(3) symptoms of hyperglycemia (described below), and a “casual” plasma glucose of greater than or equal to 11.1 mmol/L;
(4) glycated hemoglobin (a.k.a. hemoglobin A1C or HbA1C) of greater than or equal to 6.5%.
   In general, the measurements may be, and preferably are, repeated on more than one day for a definitive diagnosis of diabetes. Hyperglycemia is a condition of high plasma glucose. Symptoms of hyperglycemia include increased thirst and urination, increased hunger, blurred vision, feelings of weakness, weight loss, and dry mouth. Those people in which at least one of the following apply, (1) a fasting blood glucose that is 5.6 to 6.9 mmol/liter (100 to 125 mg/dL), and (2) a glucose tolerance test plasma glucose level of 7.8 to 11.1 mmol/liter (140 to 200 mg/dL), are classified as “pre-diabetic.”

As used herein, a “diabetic patient” is an individual (animal, including human) who has been diagnosed as having diabetes, either type 1 or type 2, or an individual, although not diagnosed as diabetic, who would be diagnosed as a diabetic individual if that individual were to be evaluated. As an example, for a human, if the plasma glucose or HbA1C, if measured, were to fall within the range described above that is classified as diabetic, that individual would be classified as a “diabetic patient,” even if not formally diagnosed. Different criteria may apply to individuals of different species. The methods of the present invention encompass treatment, prevention, and/or amelioration of vascular diseases, disorders, and conditions of those individuals classified as diabetic under current clinical criteria, as well as those who classify as diabetic under any criteria as revised or developed in the future. Those referred to as “pre-diabetic” individuals would be determined analogously.

It is believed that there are a number of reasons that diabetics exhibit higher rates of cardiovascular disease. Diabetics suffer from endothelial dysfunction making diabetics more prone to vascular lesions. The high blood glucose levels may damage heart muscle, and increase oxidative stress. Many diabetic patients have “atherogenic dyslipidemia,” or an abnormal lipid profile in the blood. This abnormal lipid profile is characterized by elevated triglycerides, and low levels of high density lipoprotein (HDL) cholesterol. Even if the low density lipoprotein (LDL) cholesterol, also referred to as “bad cholesterol,” is at a normal level, the actual LDL particles are often abnormal, such as by being smaller, denser, or both smaller and denser, and as a result, more likely to lead to atherosclerosis. Inflammation also plays a role in the development of diabetes, and plasma levels of inflammatory molecules and adhesion molecules are elevated in diabetic patients. In fact, some have referred to type II diabetes as a “chronic inflammatory disease.” At least one study has found a correlation between blood markers of inflammation and the propensity to become diabetic, but the correlation was not applicable to African Americans and smokers. In addition, animal models have shown that T cells and macrophages, both involved in immune response, are involved in the development of diabetes or insulin resistance.

Vascular diseases may also involve inflammatory processes. It is believed that the atherosclerosis plaque formation initiates with the stimulation of VCAM-1 (vascular cell adhesion molecule-1) by endothelial cells in the wall of the artery. “Atherosclerosis” refers to the depositing of fatty substances, cholesterol, cellular waste products, calcium and fibrin on the inner lining, or intima, of an artery. Smooth muscle cell proliferation and lipid accumulation accompany the deposition process. Stimulation of VCAM-1 is thought to occur by oxidized lipids. Another pathway for stimulation of VCAM-1 involves nuclear factor-κB. VCAM-1 may also be stimulated by proinflammatory cytokines Cytokines are small cell-signaling proteins. An example of a proinflammatory cytokine that may stimulate VCAM-1 is IL-1β, interluenkin-1β. VCAM-1 may also be stimulated by a substance called TNF-α, tumor necrosis factor-α. Specifically, the stimulation of VCAM-1 results in the adhesion of white blood cells, including immune modulated white blood cells. The white blood cells within the vessel wall eventually become macrophages, which are involved in immune response by engulfing and digesting cellular debris and pathogens. In the development of atherosclerosis, the macrophages engulf modified lipoproteins in the blood, particularly LDL. In a cascade effect, the macrophages also produce growth factors and cytokines, which are proinflammatory, thus attracting more white blood cells. Eventually the macrophages become the foam cells seen in atherosclerotic plaque.

Atherosclerotic plaque, also called fibrous (atheromatous) plaque and an atherosclerotic lesion, result from the accumulation of substances on the intima and occlude the lumen of the artery, a process called stenosis. When the stenosis becomes severe enough, the blood supply to the organ supplied by the particular artery is depleted resulting in a stroke, if the afflicted artery is a carotid artery, a heart attack if the artery is a coronary artery, or a loss of organ function if the artery is peripheral.

Stenting and PTCA can injure the vessel wall, such as by causing endothelial denudation, and the injury may cause inflammation. Inflammation may result in changes to smooth muscle cells, with over-proliferation of muscle cells and migration of these cells into the intima. It is the overgrowth of cells that may lead to restenosis. Thus, the vascular injury caused by stenting may eventually lead to restenosis.

Because diabetics suffer from endothelial dysfunction and inflammation, diabetics may be particularly susceptible to restenosis. It is interesting to note that the risk factors for cardiovascular disease and diabetes significantly overlap.

Embodiments of the present invention include methods of treating, preventing, or ameliorating a vascular disease and/or disorder in a diabetic or a pre-diabetic patient who is in need of treatment, prevention, or amelioration of a vascular disease and/or disorder. The methods include, but are not limited to, implanting a stent in a vascular region in a diabetic or pre-diabetic patient, and during the implantation procedure, administered (delivering) a drug formulation from a source other than the stent to the vascular region. The drug formulation may include dexamethasone, paclitaxel, or a combination thereof. It is believed that inflammation of the vessel wall may be reduced, limited, prevented or any combination thereof by the administration of a drug locally to the vascular region. In some embodiments, the patient is identified as having diabetes or a pre-diabetic condition.

Vascular regions or sites that may benefit from treatment include, but are not limited to, vascular lesions, atherosclerotic lesions, site of vulnerable plaque(s), and the site of a peripheral arterial disease. A peripheral artery disease site may be an atherosclerotic lesion in a peripheral artery that is also caused by the buildup of fatty deposits on the lining or intima of the artery walls. Examples of vascular lesions include, without limitation, saphenous vein graft lesions, restenotic lesions, bifurcation lesions, ostial lesions, left main lesions, chronic total occlusions and occlusions associated with AMI (Acute Myocardial Infarction), and STEMI (ST-segment Elevation Myocardial Infarction).

“Vulnerable plaque” refers to an atheromatous plaque that has the potential of causing a thrombotic event (formation of a clot within the vessel that blocks the vessel), and is usually characterized by a very thin wall separating it from the lumen of an artery. The thinness of the wall renders the plaque susceptible to rupture. The walls are formed from collagen which may be negatively impacted by inflammation as well as other substances present in the blood stream. When the plaque ruptures, the inner core of usually lipid-rich plaque is exposed to blood, with the potential of causing a fatal thrombotic event through adhesion and activation of platelets and plasma proteins to components of the exposed plaque.

Drugs may be administered locally or systemically. Systemic delivery involves the administration of a drug at a discrete location followed by the dispersal of the drug throughout the patient's body including, of course, to the target treatment site or organ. Local delivery is administration of the drug in such a manner as to avoid, or to substantially limit, the dispersion of the drug throughout the body. Local delivery is delivery in such a manner to concentrate the drug at the target treatment site or in the target organ, such as, for example, but not limited to, administration directly to the target site. Non-limiting examples of systemic administration include oral administration and intravenous administration. Non-limiting examples of local administration include use of an implant containing a drug, such as a drug coated stent, use of a drug coated balloon, injection by a balloon needle, or injection by a catheter.

In embodiments of the present invention, the drug is administered locally to or proximally to the vascular region of the patient. The local administration may be intra-arterial such as by a catheter, balloon catheter, a guide catheter, a micro-catheter, or an introducer sheath. A needle catheter, that is a catheter having a needle for injection may deliver the drug formulation into the adventitial space, into the vascular region to be treated such as a vascular lesion, to the surface of the vessel wall, and/or into the bloodstream. In general, the formulation may be delivered to the surface of the vessel wall and/or into the bloodstream proximally to or at the vascular region to be treated.

In some embodiments, the drug formulation is administered or delivered using a specially designed catheter or medical device that allows for occlusion of the vessel, such as a blood vessel, and then drug administration (drug delivery) occurs during the time period of occlusion. After a limited duration of time, the drug administration is stopped and the occlusion is removed allowing the lumen to open again. This process may be performed repeatedly or cycled, and in some embodiments, there are at least two cycles. The occlusion in each cycle is limited in duration of time but the total duration of occlusion may be increased by performing several cycles.

Other means of local administration may be used.

The drug formulation may be administered during the stent implantation procedure. As used herein, “during the stent implantation procedure” means that the administration of the drug formulation occurs in the same operation as the stent implantation, and typically within a time period of 6 hours or fewer. The administration may be by a bolus administration or injection, or the administration may occur by an infusion over a specific time period. The drug may be administered by an intermittent infusion. An example of intermittent infusion includes, but is not limited to, an infusion over 2 minutes, followed by 5 minutes of no drug administration, and then a subsequent infusion over 2 minutes.

The drug formulation may be administered before the stent delivery device carrying the stent is inserted into the patient, during the same time that the stent delivery device is inserted in the patient, after the stent delivery device has been removed from the patient, or a combination thereof. The drug formulation may be administered, or the administration may begin, within 30 to 90 minutes, within 5 to 75 minutes, within 10 to 45 minutes, within 5 to 30 minutes, within 2 to 20 minutes, or within 15 minutes prior to the insertion of the stent delivery device into the patient. The drug formulation may be administered, or administration may begin, during the time that the stent delivery device is in the patient, but before, during, or after the deployment of the stent. The drug may be administered at the site of the implantation within minutes, for example within 10 minutes, within 5 minutes, or within 2 minutes of the deployment of the stent at the vascular region.

In some embodiments of the present invention, the drug administration may begin within any one of the time frames disclosed above, and may end either before or after the removal of the stent delivery device from the body of the patient. In preferred embodiments, the drug administration begins within 10 minutes of stent deployment and ends within 10 minutes after the stent has been fully deployed. In other preferred embodiments, the time period of drug administration at least overlaps the time period of stent deployment, and in more preferred embodiments, at least 30% of the time period of drug administration overlaps the time period of stent deployment. In some embodiments, at least 60% of the time period of drug administration overlaps the time period of stent deployment.

In some embodiments, the drug formulation is administered prior to the insertion of the stent delivery device into the patient's body. In some embodiments, the drug formulation is administered after the stent is implanted, and may be delivered with a needle catheter designed to inject the formulation into the vascular region, the aventitial space, or a lesion with a needle fitting between the openings in the implanted stent.

A balloon used to administer the drug formulation in the methods of this invention may be microporous. A microporous balloon comprises a thin membrane in which a large number of holes, which may be of substantially uniform size, have been created. As used herein, a “hole” is an opening or a channel in a material created by any one or more of a combination of etching, laser machining, mechanical machining, drilling, and conventional processes known by persons of ordinary skill in the art. The location of holes may be predetermined. As used herein, a “pore” is an opening or channel in a material that naturally results from the properties of the material. The location of pores may not be pre-determined. As used herein, the terms “pores” and “holes” will be used interchangeably unless expressly stated otherwise. The holes or pores in the microporous balloon can range in size from tens of nanometers to microns and can be created by a number of techniques including, but not limited to, laser drilling. Alternatively, the holes or pores may be created by ab initio synthesis. In the latter case, the membrane is synthesized in such a manner that voids, openings, or channels are left in the structure formed. A microporous balloon formed by these or any other procedure may be used.

The drug formulation may include dexamethasone and/or a dexamethasone derivative. Some non-limiting examples of pharmaceutically acceptable, pharmacologically active derivatives of dexamethasone include dexamethasone phosphate, dexamethasone acetate, dexamethasone palmitate (limethasone), dexamethasone diethylaminoacetate (SOLU-FORTE-CORTIN®), dexamethasone isonicotinate, dexamethasone tetrahydrophthalate, and dexamethasone tert-butylacetate. The drug formulation may include paclitaxel (e.g. TAXOL® by Bristol-Myers Squibb Co., Stamford, Conn.), docetaxel (e.g. TAXOTERE®, from Aventis S.A., Frankfurt, Germany), or a combination thereof. The drug formulation may include any combination of the above specifically listed drugs, and may include any of the specifically listed drugs or combination thereof in combination with another drug.

It is believed that the use of dexamethasone and paclitaxel is advantageous as each has a different mechanism of action.

The dose of dexamethasone included in the drug formulation administered may be about 3.6 g/m² to about 130.0 g/m². The dose of a dexamethasone derivative administered may be such that it is equivalent to about 3.6 g/m² to about 130.0 g/m² dexamethasone. The dose of paclitaxel may be 36 mg/m² to about 1300 mg/m². In some embodiments, the drug formulation is administered over a duration of time by infusion or intermittent administration approximating an infusion where the duration may be about two hours, preferably about an hour, and more preferably about 30 minutes.

The drug formulation may include, but is not limited to, a drug, or the formulation may include, but is not limited to a drug which is dissolved, dispersed, suspended, blended, or any combination thereof, in a liquid carrier, such as water or normal saline. The drug formulation, either with or without a liquid carrier, may include an excipient. The drug formulation may include radio-opaque agents for visualization.

The drug may be included in the drug formulation in the form of particles. In preferred embodiments, the drug is included both in the form of particles as well as included in a liquid carrier and/or viscous fluid. The drug in the drug formulation that is outside of the particles provides an initial dose of drug that is available to the tissue upon delivery, while the particles provide for some period of sustained release (several hours to days or even months).

As used herein, a “particle” simply refers to a microscopic or macroscopic fragment of material of no particular shape composed of an agglomeration of individual molecules of one or more compounds. A particle can range in size from less than one tenth of a nanometer to several millimeters. As used herein, particles include core-shell structures, such as, but not limited to, micelles, worm micelles, niosomes, liposomes, and polymersomes as well as solid structures, non-limiting example of which include q-dots (quantum dots), nanocrystals, and solid or porous bits of material of the indicated dimensions. “Micelles,” “liposomes,” “worm micelles,” and “polymersomes” all fall within the broad category of “vesicles.”

There are a number of ways of representing the average diameter of a group of particles. The average diameter can be a number average diameter, where the number average diameter=Σᵢdᵢnᵢ/Σᵢnᵢ, where nᵢ represents the number of particles with a diameter represented by dᵢ. The surface area average diameter is determined by (Σᵢfᵢdᵢ²⁾^{1/2}, and the volume average diameter is determined by (Σᵢfᵢdᵢ³)^{1/3}, where fᵢ is nᵢ/Σᵢnᵢ. The volume average is greater than the surface area average diameter, which is greater than the number average diameter. The mass or weight average diameter is the same as the volume average diameter if the density of all of the particles is the same.

As used herein, the “average diameter” of a plurality of particles refers to diameters determined by dynamic light scattering (DLS), also referred to as photo correlation spectroscopy, unless expressly stated otherwise. Dynamic light scattering determines the hydrodynamic diameter (Stokes diameter) based on diffusion measurements, and includes solvent associated with the particle. For non-spherical particles, the reported “diameter” is actually the effective diameter of a sphere with the equivalent hydrodynamic radius. The mean hydrodynamic diameter which is obtained from DLS is close to the volume-average diameter. A non-limiting example of a method for determining average diameters is International Standards Organization (ISO) 13321.

As used herein, “nano-particles” refer to particles with an average diameter from 1 nm to 10 μm.

As used herein, “micro-particles” refer to particles with an average diameter from 10 μm to about 1000 μm.

Particles are generally polydisperse, i.e., not all the same size. One measure of polydispersity is the ratio D90/D10. D90 and D10 are the diameters below which 90% and 10% of the number of particles fall for a number average diameter, or 90% or 10% of the surface area of the group of particles falls for a surface area average diameter, and the like. As used herein, unless specified otherwise, the D90 and D10 are the diameters taken from the cumulative particle size distribution as determined by DLS.

A vesicle is a sack, cavity or pouch, typically filled with a liquid or gas. As used herein a “vesicle” is a compartment completely enclosed by a layer of material, or a membrane that separates the compartment and whatever may be contained in it from the external environment. The membrane is the “outer shell” of the vesicle, and the material inside is the “core.” The core may be filled with a fluid such as a liquid, a gel, a semi-solid, or a combination thereof. The core may include a drug that is dissolved, dispersed, suspended, blended, or any combination of dissolved, dispersed, suspended, and blended in the other substances in the core. A vesicle may be of any shape or no shape, i.e., essentially amorphous. Generally, however, vesicles tend to be substantially spherical or ovoid in shape. The broad category of vesicles includes, but is not limited to, “micelles,” “liposomes,” “worm micelles,” and “polymersomes.”

As used herein, “nano-vesicles” refer to vesicles with an average diameter from 1 nm to 10 μm.

As used herein, “micro-vesicles” refer to vesicles with an average diameter from 10 μm to about 1000 μm.

A “micelle” is a spherical or a substantially spherical colloidal particle, typically of nano-particle size, spontaneously formed by many amphiphilic molecules in an aqueous medium when the Critical Micelle Concentration (CMC) is exceeded. Amphiphilic molecules have two distinct components differing in their affinity for a solute, most particularly water. The part of the molecule that has an affinity for water, a polar solute, is said to be hydrophilic. The part of the molecule that has an affinity for non-polar solutes such as hydrocarbons is said to be hydrophobic. When amphiphilic molecules are placed in water, the hydrophilic moiety seeks to interact with the water while the hydrophobic moiety seeks to avoid the water. To accomplish this, the hydrophilic moiety remains in the water while the hydrophobic moiety is held above the surface of the water in the air or in a non-polar, non-miscible liquid floating on the water. The presence of this layer of molecules at the water's surface disrupts the cohesive energy at the surface and lowers surface tension. Amphiphilic molecules that have this effect are known as “surfactants.” Only so many surfactant molecules can align as just described at the water/air or water/hydrocarbon interface. When the interface becomes so crowded with surfactant molecules that no more can fit in, i.e., when the CMC is reached, any remaining surfactant molecules will form into spheres with the hydrophilic ends of the molecules facing out, that is, in contact with the water forming the micelle corona (which may be referred to as the “outer shell”) and with the hydrophobic “tails” facing toward the center of the of the sphere. Drugs suspended in the aqueous medium can be entrapped and solubilized in the hydrophobic center of micelles. Micelles formed from relatively low molecular weight surfactants generally have a CMC that is usually quite high so that the formed micelles dissociate rather rapidly upon dilution, i.e., the molecules head for open places at the surface of the water with the resulting precipitation of the drug. Non-limiting examples of relatively low molecular weight surfactants are sodium lauryl sulfate, also referred to as sodium dodecyl sulfate, polysorbates (described below), poloxamers (described below), sorbitan monolaurate 20 (SPAN™ 20) and similar compounds, CREMOPHOR EL® (a BASF trade name of a surfactant of polyethoxylated castor oil which is produced by reaction of 35 moles of ethylene oxide with each mole of castor oil), and Brij 35 (polyoxyethylene lauryl ether, (C₂H₄O)₂₃C₁₂H₂₅OH), which is a non-ionic surfactant.

A higher CMC can be obtained by using lipids with a long fatty acid chain or two fatty acid chains, specifically phospholipids and sphingolipids, or polymers, specifically block copolymers, to form the micelles.

Polymeric micelles have been prepared that exhibit CMCs as low as 10-6 M (molar). Any micelle-forming polymer presently known in the art or as such may become known in the future may be used in the embodiments of this invention. Examples of micelle-forming polymers are, without limitation, methoxy poly(ethylene glycol)-b-poly(ε-caprolactone), methoxy poly(ethylene glycol)-b-poly(D,L-lactic acid), methoxy poly(ethylene glycol)-b-poly(lactic-co-glycolic acid), conjugates of poly(ethylene glycol) with phosphatidylethanolamine, poly(ethylene oxide)-b-poly(propylene oxide)-b-poly(ethylene oxide), poly(acrylic acid)-b-polystyrene, poly(ethylene oxide)-b-polybutadiene, poly(ethylene glycol)-b-polyesters, poly(ethylene glycol)-b-poly(L-aminoacids), poly(N-vinylpyrrolidone)-b-poly(orthoesters), poly(N-vinylpyrrolidone)-b-polyanhydrides and poly(N-vinylpyrrolidone)-b-poly(alkyl acrylates).

In addition to the classical spherical micelles described above, there are “worm micelles.” Worm micelles, as the name suggests, are cylindrical in shape rather than spherical. They are prepared by varying the weight fraction of the hydrophilic polymer block to the total block copolymer molecular weight in a hydrophilic polymer-b-hydrophobic polymer structure. Polyethylene oxide has been used extensively to create worm micelles with a number of hydrophobic polymers such as, without limitation, poly(lactic acid), poly(ε-caprolactone), poly(ethyl ethylene) and polybutadiene.

Phospholipids are molecules that have two primary regions, a hydrophilic head region comprised of a phosphate of an organic molecule and one or more hydrophobic fatty acid tails. In particular, naturally-occurring phospholipids have a hydrophilic region comprised of choline, glycerol and a phosphate and two hydrophobic regions comprised of fatty acid. When phospholipids are placed in an aqueous environment, the hydrophilic heads come together in a linear configuration with their hydrophobic tails aligned essentially parallel to one another. A second line of molecules then aligns tail-to-tail with the first line as the hydrophobic tails attempt to avoid the aqueous environment. To achieve maximum avoidance of contact with the aqueous environment, i.e., at the edges of the bilayers, while at the same time minimizing the surface area to volume ratio and thereby achieve a minimal energy conformation, the two lines of phospholipids, known as a phospholipid bilayer or a lamella, converge into a sphere and in doing so entrap some of the aqueous medium, and whatever may be dissolved or suspended in it, in the core of the sphere. The core/shell construct thus formed having a shell that is a bilayer, as compared to a monolayer of a micelle, is a “liposome.” Liposomes may be unilamellar, composed of a single bilayer, or they may be multilamellar, composed of two or more concentric bilayers. Liposomes range from about 20 nm-100 nm diameter for small unilamellar vesicles (SUVs), about 100 nm-5000 nm for large multilamellar vesicles and ultimately to about 100 microns for giant multilamellar vesicles (GMVs).

Examples of phospholipids that may be used to create liposomes are, include, but are not limited to, 1,2-dimyristroyl-sn-glycero-3-phosphocholine, 1,2-dilauroyl-sn-glycero-3-phosphocholine, 1,2-distearoyl-sn-glycero-3-phosphocholine, 1,2-dimyristoyl-sn-glycero-3-phosphoethanolamine, 1,2-dipalmitoyl-sn-glycero-3-phosphoethanolamine, 1,2-dioleoyl-sn-glycero-3-phosphate monosodium salt, 1,2-dipalmitoyl-sn-glycero-3-[phosphor-rac-(1-glycerol)]sodium salt, 1,2-dimyristoyl-sn-glycero-3-[phospho-L-serine] sodium salt, 1,2-dioleoyl-sn-glycero-3-phosphoethanolamine-N-glutaryl sodium salt and 1,1′,2,2′-tetramyristoyl cardiolipin ammonium salt. An example of a class of spingolipids that may be used in forming liposomes is ceramides, a class of compounds of (N-acyl) fatty acids derivatives of a long chain base or spingosine. Many ceramides occur naturally in animal and plant tissue. An example is C-16 ceramide is illustrated below:

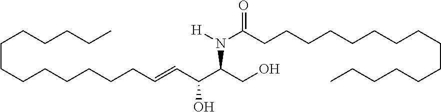 [SMILES: CCCCCCCCCCCCC/C=C/[C@@H](O)[C@H](CO)NC(=O)CCCCCCCCCCCCCCC]

A core/shell construct similar to that of a liposome but made of polymers other than phospholipids or sphingolipids is called a “polymersome.” Block copolymers may be used to form polymersomes. Depending on the length and chemical nature of the polymers in the di-block copolymer, polymersomes can be substantially more robust that liposomes. In addition, the ability to control completely the chemical nature of each block of the di-block copolymer permits tuning of the polymersome's composition to fit the desired application. For example, membrane thickness can be controlled by varying the degree of polymerization of the individual blocks. Adjusting the glass transition temperatures of the blocks will affect the fluidity and therefore the permeability of the membrane. Even the mechanism of release can be modified by altering the nature of the polymers. Some non-limiting examples of polymers that may be used to form polymersomes are poly(ethylene glycol)-b-polybutadiene, poly(ethylene glycol)-b-polyethylethylene), poly(ethylene glycol)-b-poly(ε-caprolactone), poly(ethylene glycol)-b-poly(D,L-lactic acid), and combinations of these.

“Liposomes” formed from non-ionic surfactants may also be referred to as “niosomes.”

Similar to the situation with micelles, drugs that are dissolved, dispersed, suspended, or any combination of dissolved, dispersed, and suspended in an aqueous solution may be encapsulated in the core of liposomes, and polymersomes. Methods of forming liposomes and polymersomes are known in the art. One manner of forming liposomes is emulsion templating (Pautot, et al., Langmuir, 2003, 19:2870). Polymersomes may be force-loaded by osmotically driving the drug into the core of the vesicle. One manner of forming polymersomes is using microfluidic techniques to for polymersomes from double emulsions. (Lorenceau, et al., Langmuir, 2005, 21:9183-86).

Particles may be essentially solid nano-particles or micro-particles or porous nano-particles or micro-particles. Particles may have drugs mixed, dispersed, dissolved, any combination of mixed, dissolved, and dispersed, or otherwise incorporated in the particle material. Micro-particles and nano-particles can be made of any biocompatible material including, but not limited to, natural polymer, semi-synthetic polymer, synthetic polymers, metals, ceramics, glasses, and combinations thereof. The particle material can be biostable, or biodegradable. Particles may be formed from a combination of biostable and biodegradable materials. If biodegradable materials are used in the production of particles, the particles may degrade in days, weeks, or months.

Polymeric particles with drug distributed throughout may be referred to as matrix type or monolithic type drug delivery particles. The drug may be distributed homogeneously, or substantially homogeneously, throughout the matrix particle, or the drug may be distributed non-uniformly. The drug may be released by any number of mechanisms. In some embodiments, the material dissolves, and the drug may be released as it does so. In other embodiments, the drug may diffuse through the matrix material, diffuse through pores formed when the drug is dissolved from the matrix closer to the surface, or a combination thereof.

The particles can also encapsulate drug by having an outer shell of polymer, metal, glass, ceramic, or a combination thereof, with an inner compartment (core) containing the drug, with or without other materials. The outer shell may be in the form of a coating disposed over at least a portion of the outer surface of the core of the particle. The shell and core of a particle may differ in the type of and/or the ratio of materials used to form the shell and the core of the particle. If the outer shell does not contain the drug, it may serve as a rate-controlling membrane as the drug must diffuse through the membrane. In still other embodiments, the exterior coating or layer may dissolve, biodegrade, or both dissolve and biodegrade over time resulting in release of the drug. The drug may diffuse through the membrane, and at the same time, the membrane may biodegrade, dissolve, or both biodegrade and dissolve. In some embodiments, the core if free or essentially free of drugs, and the drug is incorporated in the outer shell. The outer shell may comprise multiple layers.

The particles may be porous and the drug may be incorporated in at least some of the pores.

The particles may include combinations of the above features, and more than one drugs with different drugs incorporated in different manners. For example the particles may be porous particles and drug may be in some or substantially all of the pores, and drug may also be dispersed homogeneously within the particle material. As another example, one drug may be incorporated within the particle material and a second drug incorporated within a coating disposed over at least a portion of the surface of the particle.

There are various well-known methods by which the solid matrix particles or core-shell type particles can be fabricated including, without limitation, emulsion solvent evaporation methods, phase separation methods, interfacial methods, extrusion methods, molding methods, injection molding methods, heat press methods, coating or layering processes, spray drying, electrospraying, membrane emulsion and precision particle fabrication.

In preferred embodiments, the polymers that may be used to form nano-particles and/or micro-particles include, but are not limited to, poly(lactides), poly(lactide-co-glycolides), and combinations thereof.

In preferred embodiments, the drug formulation includes, but is not limited to, particles including drug, that are suspended or dispersed in a biocompatible solvent or vehicle (a fluid).

The drug formulation may be a viscous fluid, that is one having a viscosity, as measured at about the normal body temperature of the diabetic patient, which is about 37° C. for a human being, of about 20 cP to about 75,000 cP (centiPoise), preferably 30 cP to 40,000 cP. The viscous fluid may include the drug, dissolved, dispersed, suspended, or any combination thereof, in a biocompatible solvent or vehicle, along with an excipient. Typically the solvent will be water or a normal saline solution. Other liquid excipients, solvents, or vehicles that may be used, either individually or in combination, include, without limitation, n-methyl-2-pyrrolidone, 2-pyrrolidone, propylene glycol, ethanol, and glycerin. The liquid excipients may be used, either individually, or in combination, with water, with saline, or with normal saline.

The excipients added to the drug formulation may act as viscosity modifiers, and examples of these excipients include, without limitation, soybean oil, high fructose corn syrup, corn syrup, coconut oil, other vegetable based oils, alginic acid, chitosan, gelatin, guar gum, poly(vinyl pyrrolidone), carboxy methyl cellulose, methyl cellulose, hydroxypropyl methylcellulose, carboxymethyl cellulose, ethyl cellulose, hyaluronic acid, poly(vinyl alcohol), maltodextrins, sugars (including, but not limited to, glucose, dextrose, sucrose, trehalose, sorbitol, and xylitol), poly(ethylene glycol), xanthan gum, TWEEN™ 60 (polysorbate 60), Vitamin E TGPS, PLURONIC® F68, PLURONIC® F127, Poloxamer 407, ascorbyl palmitate, lecithins, egg yolk phospholipid, phosphatidylcholine, polyethylene glycol-phosphatidyl ethanolamine conjugate (PEG-PE), polyethylene glycol, poly(ethylene oxide), poly(vinyl alcohol), triglycerides, diglycerides, monoglycerides, fatty alcohols such as, but not limited to, aliphatic alcohols having a chain of 8 to 22 carbon atoms, and all combinations thereof. Preferred excipients for formation of a viscous formulation include poly(vinyl alcohol), hydroxypropyl methylcellulose, carboxymethyl cellulose, hyaluronic acid, polyvinylpyrrolidone, polyethylene glycol, and polyethylene oxide. Most preferred excipients are different molecular weights of polyvinylpyrrolidone is most preferred. Vitamin E TPGS is also known as D-alpha tocopheryl polyethylene glycol 1000 succinate, and is a water soluble form of Vitamin E. A specification for Vitamin-E TPGS is listed in the United States National Formulary (NF). Polysorbates are a group of oleate esters of sorbitol and its' anhydrides condensed with polymers of ethylene oxide. Polysorbates are used as emulsifiers and surfactants in food, pharmaceuticals and cosmetics. Examples include polysorbate 20, polysorbate 60, and polysorbate 80, the specifications of which are all listed in the United States Pharmacopeia (USP). PLURONIC® is a trade name of BASF and encompasses a group of block copolymers formed from ethylene oxide and propylene oxide. Poloxamers are block copolymers with a central block of poly(propylene oxide) (PPO) and with a block of poly(ethylene oxide) (PEO) on each side where the PEO blocks are usually of the same length as determined by the number of constitutional units. Poloxamer of type 407 is specified by a monograph in the National Formulary. Many of the PLURONIC® polymers are surfactants, and some of them also comply with one of the NF monographs for Poloxamers. Other copolymers and block copolymers (and co-oligomers and block co-oligomers), those that include ethylene oxide as a monomer, poly(ethylene oxide) as a block, polyethylene glycol as a block, or combinations thereof, also may be used.

In some embodiments, the drug formulation used is ABRAXANE® Paclitaxel Injection (Celegene Corporation), an albumin bound paclitaxel suspension used individually or in combination with another drug formulation and/or with excipients. In some embodiments, the drug formulation used is ORTHOVISC® (Anika Therapeutics, Inc.) used individually, or in combination with another drug formulation or with other excipients.

Excipients used in any of the drug formulations described above are preferably biodegradable, of a sufficiently low molecular weight (not more than 40,000 Dalton) to pass through the kidneys, or a combination thereof.

In some embodiments of the present invention, the stent implanted may be a stent that does not include a drug, such as a bare metal stent or a bioabsorbable polymeric stent, while in other embodiments the stent is a DES. The stent, whether it contains drug or not, may be formed from materials that are metallic, polymeric, glass, ceramic, or a combination thereof, and the materials may be biostable or biodegradable. The stent may be formed from a combination of biostable and biodegradable materials. The stent may have a coating disposed over at least a portion of the outer surface, or covering all, or substantially all, of the outer surface of the stent where the coating includes, but is not limited to including, a polymer, other material, or a combination thereof. If the stent is a DES, the coating may include a drug. The DES may be made from a biodegradable material incorporating the drug, such as, but not limited to, a polymer in which the drug is dispersed, dissolved, or a combination of dissolved and dispersed. The DES may be porous, may be hollow, or both, and the drug may be included within at least a portion of the pores, the hollow interior of the stent, or both. The DES may have cavities, indentations, grooves, or a combination thereof in the outer surface, and at least a portion of these may include the drug. The DES may include any logical combination of the above features. In some embodiments, the DES coating may be formed from coating materials free of drugs, but a drug in the device body of the DES may migrate into the coating.

If a DES is used, the drug may be, without limitation, rapamycin (sirolimus), Biolimus A9, deforolimus, AP23572 (Ariad Pharmaceuticals), myolimus, tacrolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, 40-epi-(N1-tetrazolyl)-rapamycin, or any combination thereof.

Polymers that may be used to in the preparation of particles, whether the particles are porous, solid, or of a core-shell structure such as vesicles, used in the formation of a coating disposed over at least a portion of the outer surface of a stent, used to form a material from which the device body of a stent or a portion of a stent is made, used to form the device body of a stent, or used to form a portion of a stent include, but are not limited to, poly(N-acetylglucosamine) (Chitin), Chitosan, poly(-hydroxyvalerate), poly(lactide-co-glycolide), poly(-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers include ethylene vinyl alcohol copolymer (commonly known by the generic name EVOH or by the trade name EVAL™), poly(butyl methacrylate), poly(vinylidene fluoride-co-hexafluoropropene) (e.g., SOLEF® 21508, available from Solvay Solexis PVDF, Thorofare, N.J.), polyvinylidene fluoride (otherwise known as KYNAR™, available from Atofina Chemicals, Philadelphia, Pa.), ethylene-vinyl acetate copolymers, poly(vinyl acetate), styrene-isobutylene-styrene tri-block copolymers, polyethylene glycol, and combinations thereof. The above polymers, whether used individually or in combination, may also be used in combination with other materials, such as, without limitation, metal, metal alloys, glass, ceramics, and combinations thereof.

As used herein, “lactide” encompasses L-lactide, D, L-lactide, D-lactide, meso-lactide, and any combination thereof, unless a type is specifically recited.

As used herein, the terms poly(D,L-lactide), poly(L-lactide), poly(D,L-lactide-co-glycolide), and poly(L-lactide-co-glycolide) are used interchangeably with the terms poly(D,L-lactic acid), poly(L-lactic acid), poly(D,L-lactic acid-co-glycolic acid), and poly(L-lactic acid-co-glycolic acid), respectively.

Various non-limiting embodiments of the present invention are described in the following numbered paragraphs, paragraphs (1) to (36):

(1) A method of treating, preventing, or ameliorating a vascular disease and/or disorder in a diabetic or pre-diabetic patient, the method including, but not limited to, implanting a stent in a vascular region in the diabetic patient, and during the implantation procedure, administering a drug formulation from a source other than the stent to the vascular region, wherein the drug formulation includes, but is not limited to, dexamethasone, paclitaxel, or a combination thereof; wherein the patient is in need of treating, preventing, or ameliorating a vascular disease and/or disorder; and wherein implanting the stent includes, but is not limited to, delivery of the stent to the vascular region by a stent delivery device and deployment of the stent at the vascular region.

(2) The method as described in paragraph (1), wherein administration of the drug formulation includes, but is not limited to, delivery by the use of a balloon catheter, a guide catheter, a needle catheter, or a microporous balloon catheter.

(3) The method as described in paragraph (1) or (2), wherein the administration of the drug formulation includes, but is not limited to, at least two cycles of occluding the vessel with the vascular region to be treated with drug formulation administration to the vascular region during the occlusion, and then a time period of no occlusion and no drug formulation administration.

(4) The method as described in any one of paragraphs (1)-(3), wherein the drug includes, but is not limited to, paclitaxel and the dose of the paclitaxel in the drug formulation is about 36 mg/m² to about 1300 mg/m^{2,}.

(5) The method as described in any one of paragraphs (1)-(4), wherein the drug includes, but is not limited to, dexamethasone (or a dexamethasone derivative) and the dose of the dexamethasone in the drug formulation is about 3.6 g/m² to about 130 g/m² dexamethasone.

(6) The method as described in any one of paragraphs (1)-(5), wherein the stent does not include a drug.

(7) The method as described in paragraph (6), wherein the stent is a bare metal stent.

(8) The method as described in any one of paragraphs (1)-(5), wherein the stent does include, but is not limited to including, a drug.

(9) The method as described in paragraph (8), wherein the drug of the DES is selected from the group consisting of rapamycin (sirolimus), Biolimus A9, deforolimus, AP23572 (Ariad Pharmaceuticals), tacrolimus, myolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, 40-epi-(N1-tetrazolyl)-rapamycin, and combinations thereof.

(10) The method as described in any one of paragraphs (1)-(9), wherein the drug formulation includes, but is not limited to, nano-particles, micelles, nano-vesicles, polymersomes, or any combination thereof which carry and deliver dexamethasone, paclitaxel, or a combination thereof.

(11) The method as described in paragraph (10), wherein the drug formulation includes, but is not limited to, nano-particles, the nano-particles comprising poly(lactide), poly(lactide-co-glycolide), or a combination thereof

(12) The method as described in paragraph (10), wherein the drug formulation includes, but is not limited to, nano-vesicles, the nano-vesicles being liposomes, liposomes with ceramide, or both.

(13) The method as described in paragraph (10), wherein the drug formulation includes, but is not limited to, micelles.

(14) The method as described in paragraph (10), wherein the drug formulation includes, but is not limited to, polymersomes.

(15) The method as described in any one of paragraphs (1)-(14), wherein the drug formulation includes, but is not limited to, a fluid which carries and delivers dexamethasone, paclitaxel, or a combination thereof.

(16) The method as described in in paragraph (15), wherein the fluid is a viscous fluid.

(17) The method as described in paragraph (16), wherein the drug formulation includes, but is not limited to, poly(vinyl alcohol), hydroxypropyl methyl cellulose, carboxymethyl cellulose, hyaluronic acid, polyvinylpyrrolidone, polyethylene glycol, polyethylene oxide, and combinations thereof.

(18) The method as described in any one of paragraphs (1)-(17), wherein the drug formulation is administered by intermittent administration.

(19) The method as described in any one of paragraphs (1)-(18), wherein the drug formulation is administered by a bolus administration.

(20) The method as described in any one of paragraphs (1)-(18), wherein the drug formulation is administered by an infusion.

(21) The method as described in any one of paragraphs (1)-(20), wherein the drug formulation is administered and/or the administration begins within 30 to 90 minutes prior to the insertion of the stent delivery device into the patient.

(22) The method as described in any one of paragraphs (1)-(21), wherein the drug formulation is administered and/or the administration begins within 5 to 75 minutes prior to the insertion of the stent delivery device into the patient.

(23) The method as described in paragraph (22), wherein the drug formulation is administered and/or the administration begins within 10 to 45 minutes prior to the insertion of the stent delivery device into the patient.

(24) The method as described in paragraph (22), wherein the drug formulation is administered and/or the administration begins within 5 to 30 minutes prior to the insertion of the stent delivery device into the patient.

(25) The method as described in any one of paragraphs (1)-(20), wherein the drug formulation is administered and/or the administration begins within 2 to 20 minutes prior to the insertion of the stent delivery device into the patient.

(26) The method as described in any one of paragraphs (1)-(20), wherein the drug formulation is administered and/or the administration begins within 15 minutes prior to the insertion of the stent delivery device into the patient.

(27) The method as described in any one of paragraphs (1)-(26), wherein the drug formulation is administered and/or the administration begins during the stent deployment.

(28) The method as described in any one of paragraphs (1)-(27), wherein the drug formulation is administered after the stent deployment.

(29) The method as described in any one of paragraphs (1)-(20), wherein the drug formulation is administered within 10 minutes of the deployment of the stent at the vascular region.

(30) The method as described in any one of paragraphs (1)-(20), (27) and (28), wherein the time period of drug formulation administration at least partially overlaps the time period of stent deployment.

(31) The method as described in paragraph (30), wherein at least 30% of the time period of drug formulation administration overlaps the time period of stent deployment.

(32) The method as described in paragraph (31), wherein at least 60% of the time period of drug formulation administration overlaps the time period of stent deployment.

(33) The method as described in any one of paragraphs (1)-(32), wherein the duration of drug formulation administration is about 60 minutes or less.

(34) The method as described in paragraph (33), wherein the duration of drug formulation administration is about 30 minutes or less.

(35) The method as described in any one of paragraphs (1)-(34), wherein the vascular disease in the patient is a stenosis or a restenosis.

(36) The method as described in any one of paragraphs (1)-(35), wherein the patient is identified as having a diabetic condition or a pre-diabetic condition.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention. Moreover, although individual aspects or features may have been presented with respect to one embodiment, a recitation of an aspect for one embodiment, or the recitation of an aspect in general, is intended to disclose its use in all embodiments in which that aspect or feature can be incorporated without undue experimentation.

1. A method of treating, preventing, or ameliorating a vascular disease and/or disorder in a diabetic or pre-diabetic patient, the method comprising:
implanting a stent in a vascular region in a diabetic or a pre-diabetic patient;
and
during the implantation procedure, administering a drug formulation from a source other than the stent to the vascular region, wherein the drug formulation comprises dexamethasone, paclitaxel, or a combination thereof;
wherein the patient is identified as having a diabetic condition or a pre-diabetic condition;
wherein the patient is in need of treating, preventing, or ameliorating a vascular disease and/or disorder;
and
wherein implanting the stent comprises delivery of the stent to the vascular region and deployment of the stent at the vascular region.

2. The method of claim 1, wherein the drug formulation administration comprises administration by a balloon catheter, a catheter, a needle catheter, a microporous balloon, and/or a guide catheter.

3. The method of claim 1, wherein administration of the drug formulation comprises administration of paclitaxel at a dose of about 36 mg/m² to about 1300 mg/m².

4. The method of claim 1, wherein administration of the drug formulation comprises administration of dexamethasone at a dose of about 3.6 g/m² to about 130.0 g/m² dexamethasone.

5. The method of claim 1, wherein the stent does not comprise a drug.

6. The method of claim 5, wherein the stent is a bare metal stent.

7. The method of claim 1, wherein the drug formulation comprises nano-particles, micelles, nano-vesicles, polymersomes, or any combination thereof which carry and deliver the dexamethasone, paclitaxel, or a combination thereof.

8. The method of claim 7, wherein the drug formulation comprises nano-particles, the nano-particles comprising poly(lactide), poly(lactide-co-glycolide), or a combination thereof.

9. The method of claim 7, wherein the drug formulation comprises nano-vesicles, the nano-vesicles being liposomes, liposomes with ceramide, or both.

10. The method of claim 7, wherein the drug formulation comprises micelles.

11. The method of claim 7, wherein the drug formulation comprises polymersomes.

12. The method of claim 1, wherein the drug formulation comprises a viscous fluid which carries and delivers the dexamethsaone, paclitaxel, or a combination thereof.

13. The method of claim 12, wherein the drug formulation comprises poly(vinyl alcohol), hydroxypropyl methyl cellulose, carboxymethyl cellulose, hyaluronic acid, polyvinylpyrrolidone, polyethylene glycol, polyethylene oxide, or a combination thereof.

14. The method of claim 1, wherein the stent is a drug eluting stent.

15. The method of claim 14, wherein the drug of the drug eluting stent is selected from the group consisting of rapamycin (sirolimus), Biolimus A9, deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, 40-epi-(N1-tetrazolyl)-rapamycin, and combinations thereof.

16. The method of claim 1, wherein the drug formulation is administered by a bolus administration, by an infusion, or by intermittent administration.

17. The method of claim 1, wherein the drug formulation is administered by infusion.

18. The method of claim 1, wherein the drug formulation is administered and/or the administration begins within 30 to 90 minutes prior to the insertion of the stent delivery device into the patient.

19. The method of claim 1, wherein the drug formulation is administered and/or the administration begins within 5 to 75 minutes prior to the insertion of the stent delivery device into the patient.

20. The method of claim 1, wherein the drug formulation is administered and/or the administration begins within 15 minutes prior to the insertion of the stent delivery device into the patient.

21. (canceled)

22. The method of claim 1, wherein the drug formulation is administered after the stent deployment.

23. The method of claim 1, wherein the time period of drug administration at least partially overlaps the time period of stent deployment.

24. The method of claim 1, wherein at least 30% of the time period of drug administration overlaps the time period of stent deployment.

25. The method of claim 1, wherein the drug administration comprises at least two cycles, each cycle comprising occluding the vessel including the vascular region and administering the drug formulation during the time period of occlusion followed by a time period of no occlusion and no drug formulation administration.

26. The method of claim 1, wherein the vascular disease in the patient is a stenosis or a restenosis.

27. The method of claim 12, wherein the drug formulation comprises hydroxypropyl methyl cellulose, carboxymethyl cellulose, or a combination thereof.
